(19) United States
(12) Patent Application Publication
(10) Pub. No.: US 2010/0130969 A1
(43) Pub. Date: May 27, 2010

(54) SYSTEM AND METHOD FOR DERMATOLOGICAL TREATMENT

(75) Inventors: Rebecca A. Batterson, Carlsbad, CA (US); Gregory C. Mooradian, San Diego, CA (US)

Correspondence Address: BURNS & LEVINSON, LLP, 125 SUMMER STREET, BOSTON, MA, 02110, US

(73) Assignee: APOGEN TECHNOLOGIES, INC., San Diego, CA (US)

(21) Appl. No.: 12/565,974

(22) Filed: Sep. 24, 2009

Related U.S. Application Data
(60) Provisional application No. 61/117,623, filed on Nov. 25, 2008.

(51) Int. Cl. A61B 18/18

(52) U.S. Cl. 606/9

(57) ABSTRACT

A system and method for dermatological treatment uses a handpiece and a control system. The treatment area is analyzed for abnormalities and a customized treatment profile is created. Electromagnetic radiation then treats abnormalities in the treatment area, which can be shaped and pixilated into different intensities according to the treatment profile.

This application claims the priority date of Provisional Application Ser. No. 61/117,623, entitled SYSTEM AND METHOD FOR DERMATOLOGICAL TREATMENT, filed on Nov. 25, 2008, which this application incorporates by reference in its entirety.

### BACKGROUND

The present teachings relate generally to electromagnetic radiation and, more particularly, to dermatological treatment devices using electromagnetic radiation. What is needed are a system and method for easy-to-use, customizable electromagnetic radiation treatment of skin abnormalities.

### SUMMARY

The needs set forth herein as well as further and other needs and advantages are addressed by the present embodiments, which illustrate solutions and advantages described below.

The system of the present embodiment includes, but is not limited to: an analysis electromagnetic radiation system that delivers analysis electromagnetic radiation to the treatment area; an image acquisition system that acquires an electronic image of the analysis electromagnetic radiation delivered to the treatment area; computer readable media having threshold treatment values; control electronics that creates a treatment profile by selecting threshold treatment values from the computer readable media using the electronic image; a display that displays the treatment profile; and a treatment electromagnetic radiation system that delivers treatment electromagnetic radiation to the treatment area according to the treatment profile.

The method of the present embodiment includes, but is not limited to: selecting a treatment type; delivering analysis electromagnetic radiation to the treatment area; capturing an electronic image of the analysis electromagnetic radiation returning from treatment area; determining a treatment profile using the electronic image and the selected treatment type; displaying the treatment profile to a user; allowing the user to adjust the treatment profile; and delivering treatment electromagnetic radiation to the treatment area according to the treatment profile.

Other embodiments of the system and method are described in detail below and are also part of the present teachings.

For a better understanding of the present embodiments, together with other and further aspects thereof, reference is made to the accompanying drawings and detailed description, and its scope will be pointed out in the appended claims

### BRIEF DESCRIPTION OF THE DRAWINGS

FIG. 1 is a pictorial illustration of one embodiment of a system for dermatological treatments;

FIG. 2 is a block diagram of one embodiment of the system;

FIG. 3A is an illustration of a non-uniform lesion;

FIG. 3B is an illustration of the treatment profile for the non-uniform lesion from FIG. 3A;

FIGS. 4A is a schematic illustration of one embodiment of the handpiece;

FIGS. 4B and 4C are schematic illustrations of skin abnormalities;

FIG. 5A is a schematic block diagram of one embodiment of the main control system;

FIG. 5B is another schematic block diagram of one embodiment of the main control system;

FIG. 6A is another schematic illustration of one embodiment of the handpiece;

FIGS. 6B and 6C are schematic illustrations of skin abnormalities;

FIG. 7A is a schematic illustration of another embodiment of the handpiece;

FIGS. 7B and 7C are schematic illustrations of skin abnormalities;

FIG. 8A is a schematic block diagram of another embodiment of the main control system;

FIG. 8B is another schematic block diagram of an embodiment of the main control system;

FIG. 9A is a schematic illustration of a another embodiment of the handpiece;

FIGS. 9B and 9C are schematic illustrations of skin abnormalities;

FIG. 10 is a flowchart depicting one embodiment of the method of dermatological treatment; and

FIG. 11 is a graphical representation of one embodiment of a histogram.

### DETAILED DESCRIPTION

The present teachings are described more fully hereinafter with reference to the accompanying drawings, in which the present embodiments are shown. The following description is presented for illustrative purposes only and the present teachings should not be limited to these embodiments.

Electromagnetic radiation including ultraviolet, visible and near infrared (NIR) light, although not limited thereto, may be used in the treatment of dermatological conditions or abnormalities such as the elimination of pigmented and vascular lesions, tattoos, acne, and unwanted hair, although not limited to these conditions. Such dermatological treatments are performed by targeting chromophores within the skin (melanin, water, hemoglobin, or ink in the case of tattoo removal) with high powered electromagnetic radiation sources that are within the appropriate chromophore absorption bands, in order to induce photothermolysis or cell necrosis. In the case of pigmented lesions, when the cells die, they either slough off or they are removed by the body's immune system. When veins are treated with an electromagnetic radiation source that is within the absorption band of hemoglobin, the veins collapse and over time are also removed by the body's immune system. The result is a fading or disappearance of the lesion, and a more uniform or desirable skin appearance.

Electromagnetic radiation treatment sources are designed to produce electromagnetic radiation at or near the peak of the particular chromophore absorption wavelengths. Pulsing of the electromagnetic radiation is required when very high levels of electromagnetic radiation must be absorbed within the relaxation time of the target chromophore. Electromagnetic radiation sources for dermatological treatments are typically lasers, but also include LED's, broadband flash lamps, and other electromagnetic radiation sources.

Although damage to skin lesions or abnormalities in the tissue is possible and desirable, damage to normal tissue is obviously undesirable. Therefore, various methods are employed to avoid normal tissue damage, which can result in burning, scarring, and unnecessary pain. Normal skin that has a relatively high melanin content also has higher susceptibility to photodamage during treatments with electromagnetic radiation within the absorption band of melanin. The risk or the amount of damage increases as the level of melanin in the skin increases.

U.S. Pat. No. 7,328,060, issued on Feb. 5, 2008, assigned to Science & Engineering Associates, Inc. and incorporated herein by reference in its entirety, discloses a system for photodynamic therapy of cancerous and/or pre-cancerous cells.

Electromagnetic radiation treatments are typically performed with relatively uniform (spatial) illumination with respect to the beam intensity profile. Uniform illumination, or the elimination of hot spots in the beam, allows the operator to better control the intensity of the treatment beam while treating the tissue. However, the lesion itself may not be uniform, so different areas of the lesion may require different dosages of electromagnetic radiation. If one intensity level of electromagnetic radiation is used across a lesion that is non-uniform (e.g., in color, etc.), over or under-treatment could result.

In addition, if the electromagnetic radiation treatment pattern is circular and the lesion is large, it may be difficult to move the beam of electromagnetic radiation from place to place across the entire lesion without either overlapping where the treatment hits the tissue (double-treating), or leaving treatment gaps. A square pattern may provide an improved ability to avoid treatment overlap or treatment gaps, but skill and a significant manual effort is required to position the beam. It is also difficult to keep track of the edges of the treated areas during treatment, so operators may purposely overlap the edges of subsequent treatments to avoid gaps. However, they risk over treatment of the double-treated areas.

If the circular beam is very small, it may provide the ability to treat all areas of the lesion without overlapping treatment beams or leaving gaps, but the smaller the beam diameter, the more steps are involved. This leads to significantly increased treatment time.

One may use a system combining a boundary and a boundary sensor to detect if the laser is directly above the lesion prior to laser activation, improving the ability to accurately target the lesion. The system may shut off the laser beam if the laser is not directly over the lesion (meaning that it is directly over normal tissue). A boundary can be made with any white material, such as adhesive tape or construction paper. This method may reduce the risk of treating normal skin, but requires a significant amount of preparation time in order to create the boundary around the lesion. In addition, erratically shaped lesions or non-uniform lesions still require significant skill to optimize treatment.

Another way to reduce the risk of damage to normal tissue may be to perform a baseline measurement of the normal tissue. A threshold level may be acquired by taking a reading of the patient's normal tissue by using reflected electromagnetic radiation to measure the skin characteristics (e.g., hemoglobin, melanin, etc.). Then, electromagnetic radiation may be applied to an area only if it is above the threshold of the normal tissue, indicating abnormalities. However, this method assumes that the tissue beneath the treatment area is either all normal or all abnormal, which can be problematic. For instance, if half of the treatment area is normal and half of the treatment area is abnormal, the detector will average the signal from the entire treatment area, which will be considered above threshold, thus treating the normal tissue area. In addition, lesions can have very irregular shapes, and any variations within a lesion could lead to under or over-treatment of some areas. If a small treatment beam is used (˜1 mm) so that the measurements can be more accurate, a lot of time and skill will be required to step the beam from point to point across the entire lesion.

Laser beams used for treatment may be controlled by using an outline to contain the beam. The outline may be drawn with an aiming beam, visible to the user before the treatment begins. But it still may be difficult to accommodate irregular shapes, which can result in normal tissue damage.

A tracking device may be used to monitor the position of a laser beam. This allows the user to maneuver the beam around a lesion. Over or under-radiation may be reduced since the beam will not deliver electromagnetic radiation where it has done so previously. While this method prevents double-treatment, it still does not prevent the unwanted treatment of normal tissue.

Indicia may be positioned around the edges of the lesion such that the laser will only fire if it is over the lesion area. But this method still requires significant preparation time and a skilled operator to manually position the indicia.

One may also use pre-defined patterns of uniform electromagnetic radiation to create beam shapes similar to the lesion being treated. Pre-defined patterns may be square, rectangular, or hexagon. Having a choice of shapes may reduce the number of steps required to treat an entire lesion, by using a shape that fits within the lesion better than a simple circle or square, but it still requires a skilled operator to manually position the device. Further, the beam must be stepped from place to place across the lesion to ensure that the treatment beams do not overlap or leave gaps. In addition, lesions typically have irregular shapes, so staying within the boundaries of the lesion can still be difficult.

Robotics may be used for the treatment of facial rejuvenation. Such systems may first map a patient's face so that lesions or other dermatological conditions can be automatically treated. A software program may be employed to make those determinations, and a robot may perform automated treatment. This may reduce the skill level required of the operator, although the robot would likely have to be calibrated with the patient's body. These robotics are also presumed to be very expensive and may have difficulties in covering all areas of the body.

A camera may be employed at the head of a laser handpiece to enhance viewing of the treatment area. However, a skilled operator would still need to manually perform the treatments.

What is needed is a way to automatically and accurately treat dermatological conditions or abnormalities which does not rely upon operator skill It is a further need to perform this treatment without significant risk of damaging normal tissue.

In one embodiment the system has optical and electronic components to automatically treat tissue lesions, abnormalities, and pre-cancers or cancers within living tissue, although not limited thereto. Tissue lesions may be pigmented lesions or vascular lesions, although not limited thereto. Abnormalities are any object within the tissue that creates a non-uniform skin appearance. The system may perform treatments in conjunction with a photosensitizing agent, although not limited thereto, so that cancerous or pre-cancerous cells are killed during treatment. The electromagnetic radiation delivery device may communicate with a main control system. This system may image the treatment area onto a detector array, determine the shape and structure of the area (e.g., lesion, abnormality, pre-cancers, cancers, etc.) to be treated, and then provide a structured electromagnetic radiation treatment beam that has the appropriate intensity profile and shape to maximize treatment. Thus, the treatment beam provides the proper amount of electromagnetic radiation throughout the treatment area, no matter how complex the shape of the lesion, abnormality, or cancerous or pre-cancerous cells, while avoiding the treatment of normal tissue. The system may use electronic images of the treatment area to adaptively and dynamically adjust the treatment light beam during treatment, since the optimum dose changes during the treatment process.

Lesions (used herein as a collective term, also refers to any other abnormalities, pre-cancers, cancers, etc.) that are within the size of the illumination area of the electromagnetic radiation delivery device may be fully treated without moving the electromagnetic radiation delivery device. For lesions that are larger than the illumination area of the electromagnetic radiation delivery device, the electromagnetic radiation delivery device may be moved across the lesion until the entire lesion is treated. Analysis software may be used to determine the proper treatment beam threshold, which differentiates between normal tissue and the tissue to be treated. Threshold values may be customized for pigmented lesions, vascular lesions, tattoos, acne, lesions already having received treatment, hair follicles, or other objects. This allows the operator to move the electromagnetic radiation delivery device from place to place across the lesion area (manually or automatically), while greatly reducing the risk of burning or scarring caused by over-treatment.

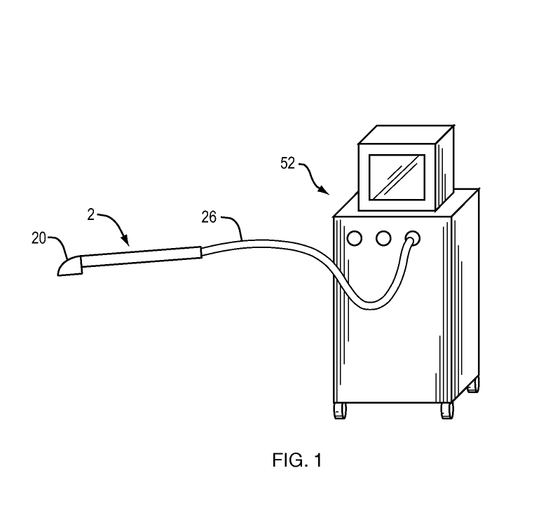

Referring now to FIG. 1, shown is a pictorial illustration of one embodiment of a system for dermatological treatments. The system may have a main control system 52 and a handpiece 2 (also referred to as “electromagnetic radiation delivery device”) to provide the necessary functionality. An operator may position the head 20 (also referred to as “treatment head”) of the handpiece 2 over the area of skin to be treated (also referred to as “treatment area,” not shown in FIG. 1). An image of the treatment area may be captured at the head 20 of the handpiece 2, described further below, and transmitted to the main control system for 52 for analysis. An illumination source (or sources) inside the handpiece 2 may provide electromagnetic radiation to the treatment area. The resulting analysis electromagnetic radiation (e.g., reflected fluorescence, autofluorescence, etc.) is used for improving the image characteristics and for tissue analysis of the treatment area. The analysis electromagnetic radiation (e.g., “image”) is transmitted to the main control system 52 through a conduit 26 (also referred to as “image conduit”). In one embodiment, the image is converted into an electronic signal before sending it to the main control system 52, although not limited to this embodiment. Therefore, the conduit 26 may be optical, electrical, or some other apparatus capable of transmitting information.

Before an image is converted into an electronic signal, it may be filtered to obtain information about the treatment area, discussed further below. The information may then be transferred to the control system 52 for analysis. Analysis software, discussed further below, may analyze the image information and determine the appropriate shape and intensity profile for an electromagnetic radiation treatment beam.

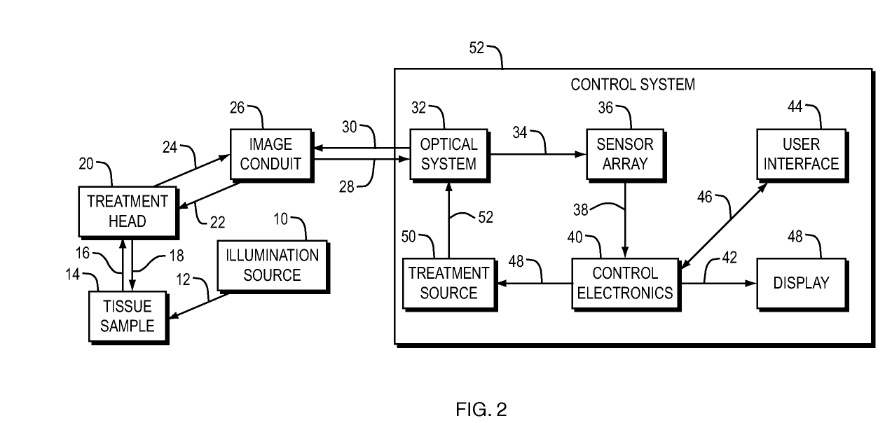

Referring now to FIG. 2, shown is a block diagram of one embodiment of the system. An illumination source 10 in the treatment head 20 may be used to improve viewing of the treatment area 14 or it can be used for tissue analysis purposes. The image of the tissue sample 14 is then transmitted to the control system 52 through the image conduit 26. The control system 52 may have a sensor array 36 to analyze characteristics of the image provided by an optical system 32. These characteristics may then be transmitted to control electronics 40 and displayed to the user on a display 48. The control electronics 40, described further below, may create a custom treatment profile, based upon any abnormalities detected within the tissue sample 14, for the treatment source 50. The treatment source 50 may transmit the treatment profile to the optical system 32 so that the electromagnetic radiation treatment may be transmitted by the treatment head 20 to the treatment area.

A system so constructed provides an easy-to-use electromagnetic radiation treatment device. Continuous analysis of the treatment area may allow a continuously updated treatment profile so that the user of the system need not be highly-skilled. By simply placing the treatment head 20 over the treatment area, the system may automatically create and administer customized electromagnetic radiation treatment. Such a system also has fewer components since the same image conduit 26 may be used to both image the tissue sample 14 as well as provide a customized treatment profile—the user only needs to place the treatment head 20 over the tissue sample to accomplish both tasks.

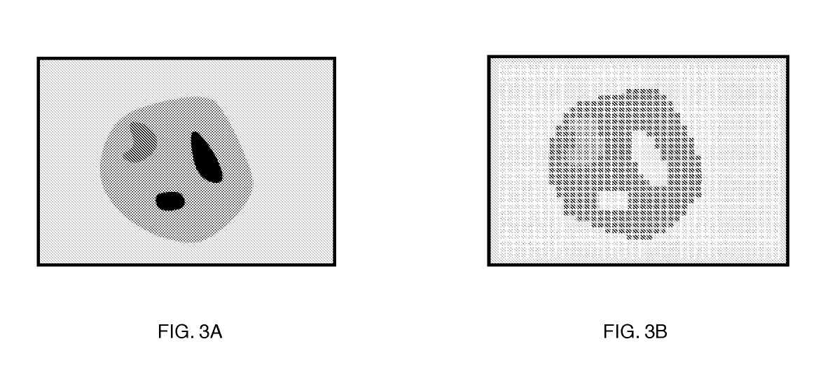

Referring now to FIG. 3A, shown is an illustration of a non-uniform lesion. The lesion may be imaged and analyzed by the system. FIG. 3B is an illustration of the treatment profile for the non-uniform lesion from FIG. 3A. The image of the lesion at the treatment area has been pixilated to demonstrate how the system controls electromagnetic radiation treatment beams on a pixel-by-pixel basis. This particular illustration demonstrates that at the darker area a lower intensity of electromagnetic radiation is needed. The actual treatment beam is capable of a much higher resolution intensity profile, and this figure is only an example of a pixilated treatment beam profile.

The illustrations shown in FIGS. 3A and 3B may be displayed to a user of the system on a display 48 (shown in FIG. 1). The display 48 may show the calculated treatment beam image (shown in FIG. 3B) overlaid onto the actual image (shown in FIG. 3A) of treatment area. An operator may use this image information to manually set the appropriate threshold for treatment. Thresholds may be set according to the tissue properties, which may be levels of darkness or color level, hemoglobin concentration, and temperature, although not limited thereto, depending on the type of lesion or skin abnormality. As the operator adjusts the threshold through the user interface 44 (shown in FIG. 2), the treatment beam shape may grow or shrink, depending upon which way the threshold is adjusted.

Once the treatment beam profile matches the shape of the lesion, the operator may initiate treatment. A low-level electromagnetic radiation beam may be projected along the treatment beam path and onto the treatment area to demonstrate the shape and positioning of treatment electromagnetic radiation at the treatment area. If the treatment beam is smaller than the treatment area, the treatment head may be stepped across the lesion. This can be performed either manually or automatically. Analysis of the imaged treatment area assures that normal skin will not be treated, so moving the handpiece from place to place across the lesion does not require significant skill.

Once the electromagnetic radiation treatment has been administered, analysis software determines if additional treatment is needed. The analysis software may be designed to include post-treatment analysis to determine which areas require additional treatment and by what amount. For photodynamic therapy treatments or applications that use a continuous light source as opposed to a pulsed source, the system will continually monitor the tissue and adjust the treatment beam characteristics accordingly.

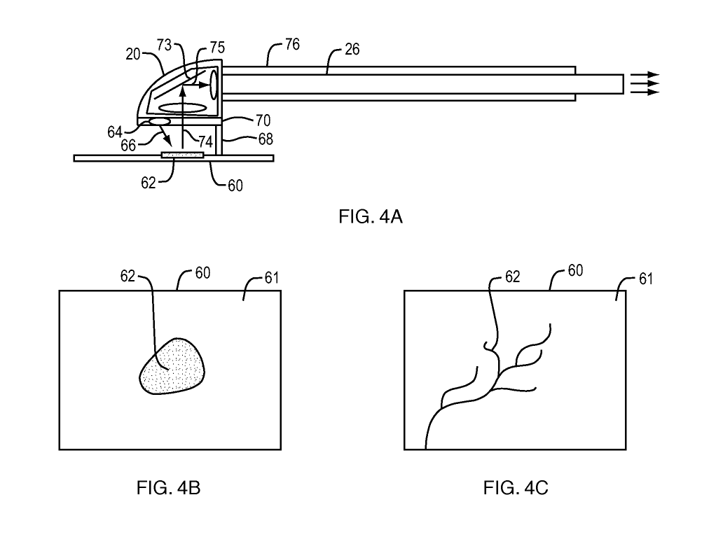

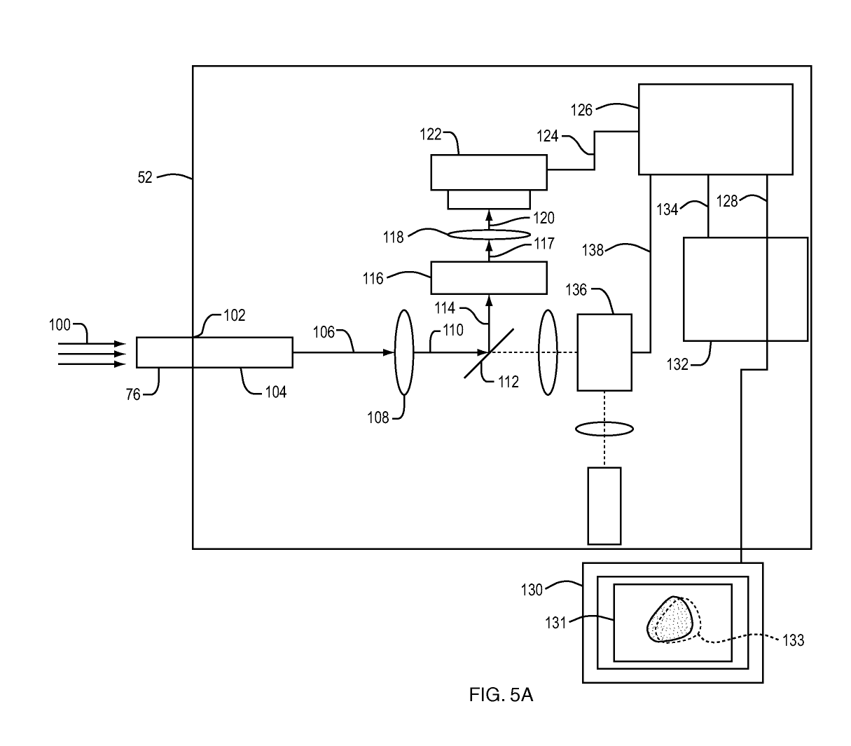

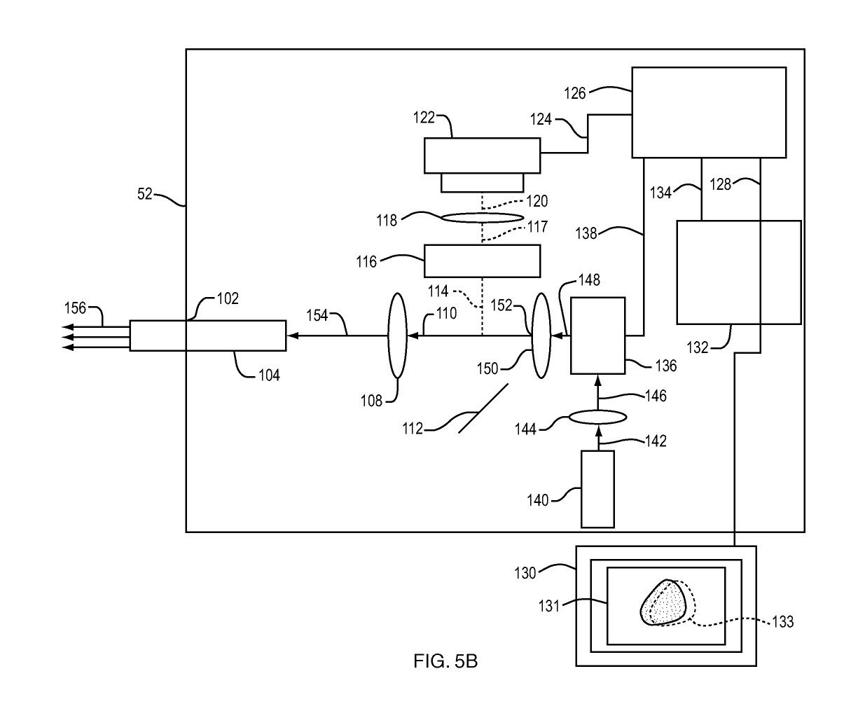

Referring now to FIG. 4A, shown is a schematic illustration of one embodiment of the handpiece 2. FIGS. 4B and 4C are schematic illustrations of skin abnormalities. In this embodiment, an image of the treatment area 60 is transmitted to the main control system 52 where a sensor array 122 is housed (shown in FIG. 5A). The treatment head 20 may be positioned so that its window 70 is centered above the treatment area 60. It is appreciated that the window 70 may be open, or may comprise glass or some other material that permits electromagnetic radiation to pass. A stand-off 68 or some similar spacing device may be employed to ensure the treatment window 70 is at a fixed distance from the treatment area 60. The stand-off 68 can be of various sizes and shapes. A fixed distance allows the image to be fixed in one plane for the imaging optics. An illumination source 64 provides electromagnetic radiation 66 to the treatment area 60 which interacts with the tissue and returns back into the treatment head 20 to improve the signal-to-noise ratio of the treatment area image. This signal may be used for visualization or tissue analysis purposes and helps to distinguish between the normal and abnormal tissue. The abnormal tissue 62 may be a pigmented lesion, a vascular lesion, a tattoo, or other dermatological abnormality. Hair or hair follicles are also detected, as these are different from normal tissue 61.

The electromagnetic radiation 66 from the illumination source 64 provides a beam of electromagnetic radiation to the treatment area 60, and resulting electromagnetic radiation (reflected, fluorescence, etc.) 74 enters into the treatment head 20 via the window 70. The resulting electromagnetic radiation 74 may travel into beam steering optics 73, which then directs the electromagnetic radiation into an image conduit 26 to be transmitted to the control system 52. The image conduit 26 may include a fiber bundle, a series of lenses and minors, or any other method of transmitting electromagnetic radiation.

Referring now to FIG. 5A, shown is a schematic block diagram of one embodiment of the main control system 52. Electromagnetic radiation 100 returning from the treatment area (also referred to as “analysis electromagnetic radiation,” shown in FIG. 4) travels through the image conduit into the control system 52 through the electromagnetic radiation delivery port 102. The electromagnetic radiation 106 exits the proximal end of the electromagnetic radiation delivery device 104 and travels through collimating optics 108. After passing through the collimating optics 108, the electromagnetic radiation 110 reflects off of a movable mirror 112 and the reflected electromagnetic radiation 114 travels through the filter optics 116. The electromagnetic radiation exiting the filter optics 117 enters relay optics 118. The combination of the collimating optics 108 and relay optics 118 creates an image on the sensor array 122 with the electromagnetic radiation beam 120 of the treatment area. A filter 116 may be used to separate different frequencies of electromagnetic radiation within the image for analysis. The filter 116 may be a multiple of filters, a prism filter design, a single filter, or no filter at all.

The sensor array 122 transfers the optical signal into an electronic signal and sends the signal 124 to the control electronics 126. The control electronics 126 creates image information 128 for the display 130. An image of the treatment area 131 may provide the operator an image on the display 130. The operator may use controls on the user interface 132 to adjust the display characteristics, the treatment threshold, and treatment parameters, although not limited to these functions. Adjustment signals 134 may be sent back to the control electronics 126.

Referring now to FIG. 5B, shown is another schematic block diagram of one embodiment of the main control system. The proposed treatment beam 133 may overlay the treatment area 131 image on the user display 130. Once the operator decides that the treatment beam parameters and shape are correct, the operator may initiate the treatment beam via the user interface 132. The analysis software, discussed further below, may analyze the treatment area and develop a customized treatment profile.

Prior to treatment, the movable mirror 112 is positioned out of the electromagnetic radiation pathway between the projection optics 150 and the collimating lens 108 in order to permit the treatment electromagnetic radiation 152 to pass through. The treatment electromagnetic radiation source 140 can be a laser, LED, broadband lamp, some other device, or a combination thereof. The electromagnetic radiation 142 from the treatment electromagnetic radiation source 140 may pass through collimating optics 144 into the beam shaping mechanism 136. The control electronics 126 sends a signal 138 to the beam shaping mechanism 136 in order to control the shape and intensity of the treatment beam. The beam shaping mechanism may be a spatial electromagnetic radiation modulator, liquid crystal display, liquid crystal shutter mechanism, or other pixilated mechanism that controls electromagnetic radiation reflection or throughput by individually turning each pixel on or off, although not limited thereto.

The amount of electromagnetic radiation that is transmitted through or reflected from each pixel may be controlled by how long the pixel is in the on or off position. The resolution of the treatment beam and intensity at each point is controlled by the beam shaping mechanism 136, discussed further below.

The electromagnetic radiation 148 from the beam shaping mechanism 136 is then transmitted through the projection optics 150. The projection beam 152 passes to the collimating optics 108 (the movable mirror is no longer in the way). The projection optics 150 and the collimating optics 108 are designed to transfer the image of the beam shaping optics 136 to the proximal end of the electromagnetic radiation delivery device 104 for treatment at the treatment area.

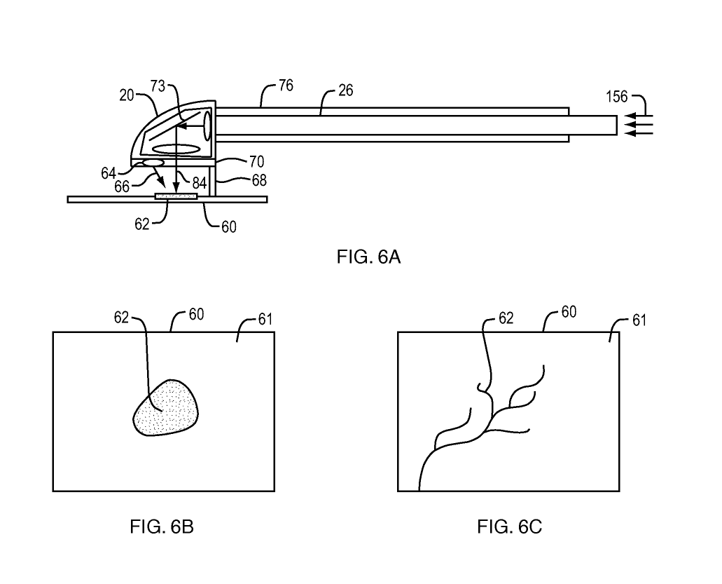

Referring now to FIG. 6A, shown is another schematic illustration of one embodiment of the handpiece. FIGS. 6B and 6C are schematic illustrations of skin abnormalities. The electromagnetic radiation 156 from the proximal end of the electromagnetic radiation delivery device 104 (shown in FIG. 5B) may be transferred through the image conduit 26 into the beam steering optics 73 inside the treatment head 20. After exiting the beam steering optics 73, the electromagnetic radiation 84 passes through the window 70 of the treatment head 20 and onto the treatment area 60. The treatment electromagnetic radiation 84 has the shape of the lesion or abnormal tissue 62 and may also have a predetermined treatment border around the abnormality. The relative intensity at each point (e.g., pixel, etc.) on the beam may be adjusted so that the treatment outcome is more uniform across the treatment area 60.

For photodynamic therapy treatments, or treatments in which the treatment electromagnetic radiation is applied for multiple seconds (or longer), the system will continually monitor the tissue and adjust the treatment beam characteristics accordingly. Since the treatment electromagnetic radiation 84 can be controlled on a pixel by pixel basis, any number of different treatment profiles may be created.

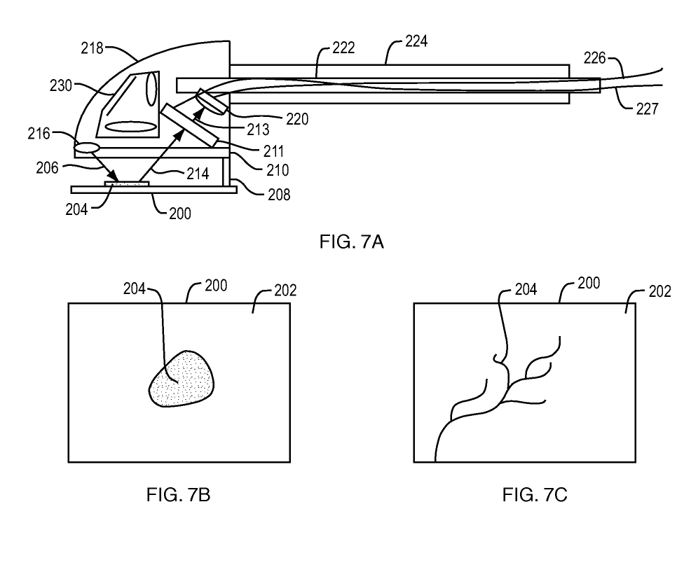

Referring now to FIG. 7A, shown is a schematic illustration of another embodiment of the handpiece 2. FIGS. 7B and 7C are schematic illustrations of skin abnormalities. As disclosed here, the detector array 220 may be housed inside the treatment head 218. The treatment head 218 is positioned so that the window 210 is centered above the treatment area 200. A stand-off 208 may be used to ensure a fixed distance between the window 210 and the treatment area 200. The stand-off can be of various sizes and shapes. The fixed distance allows the image to be fixed in one plane for the imaging optics. The illumination source 216 provides electromagnetic radiation 206 to the treatment area 200. This electromagnetic radiation interacts with the tissue within the treatment area and the resulting electromagnetic radiation (e.g., reflected fluorescence, autofluorescence, etc.) returns 214 into the handpiece 2 so that the treatment area 200 can be clearly seen by the sensor array 220. This analysis electromagnetic radiation 206 helps to differentiate between normal tissue 202 and a lesion or abnormality 204 using various image analysis methods. The source may be a laser, an LED, a flash lamp any combination of these, although not limited thereto. The abnormality may be a pigmented lesion, a vascular lesion, a tattoo, hair/hair follicles, or other dermatological abnormality (a pigmented lesion and a vascular lesion are shown 204).

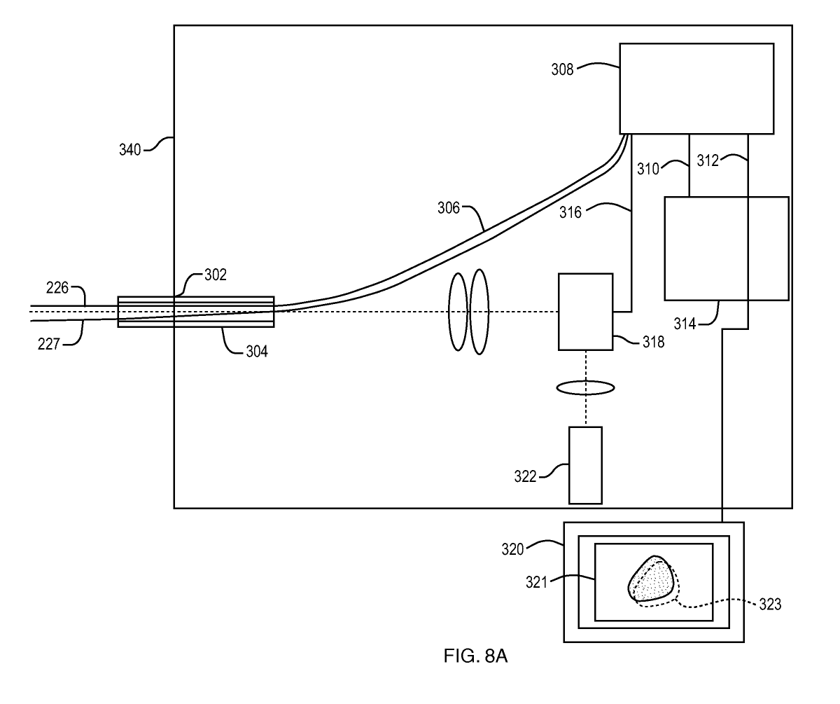

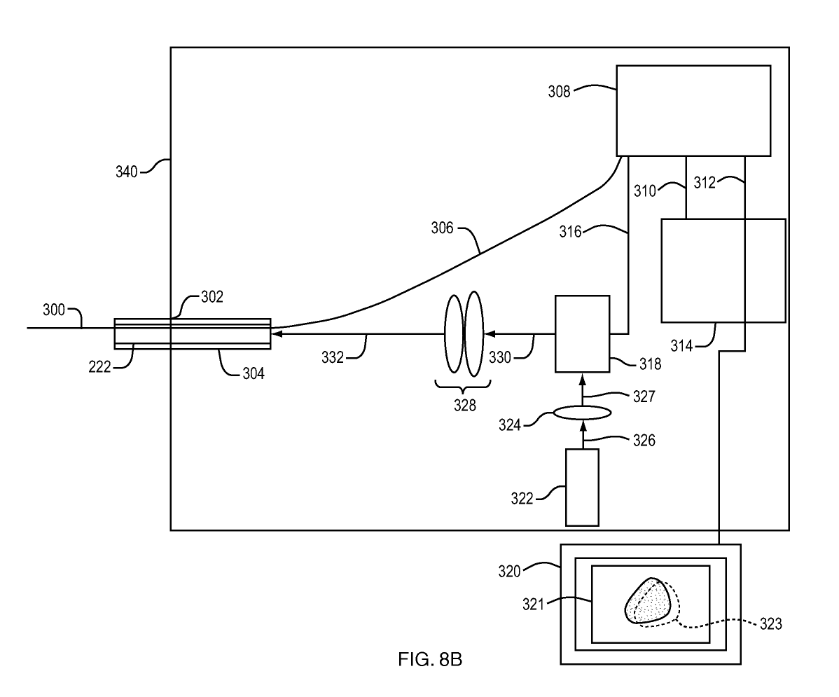

The analysis electromagnetic radiation 214 (e.g., reflected light, fluorescence, autofluorescence, etc.) enters a filter system 211 and provides image information to the detector array 220 in different optical wavebands or wavelengths for image analysis. The filter system 211 may be a multiple of filters, a prism filter design, a single filter, or no filter at all. After the filtered electromagnetic radiation 213 enters the detector array 220, it may be transmitted through an electronic cable 226 to the main control system 340 (shown in FIG. 8A). This embodiment differs from that shown in FIGS. 4, 5A, 5B and 6 where the optical image is sent to the main control system. Here, the optical image is converted to electrical information inside of the treatment head 218.

Referring now to FIG. 8A, shown is a schematic block diagram of another embodiment of the main control system. The image signal from the treatment head 218 (shown in FIG. 7) travels through the image conduit 226 and enters the control system 340 through a cable port 302. The image signal then travels to the control electronics 308 via cable 306. Cable 306 may be the same cable as 226, or a joint may be added, although not limited to this embodiment. The control electronics 308 creates image information 312 for the user display 320. The image of the treatment area 321 may be shown on the display 320. The operator may use controls on the user interface 314 to adjust the threshold or treatment parameters. The signal from the adjusted threshold or treatment parameters 310 may be sent to the control electronics 308, and the adjusted image shown on the user display 320. The proposed treatment beam 323, which may be determined by the threshold, may be projected over the treatment area 321 image on the user display 320 so that the user may easily determine the proper threshold for treatment.

The control electronics 308 may send signals 316 to a beam shaping mechanism 318, which controls the shape and intensity of the treatment beam. The beam shaping mechanism 318 can be a spatial electromagnetic radiation modulator, liquid crystal display, liquid crystal shutter mechanism, or other pixilated mechanism that controls electromagnetic radiation reflection or throughput by individually turning each pixel on or off, although not limited thereto. The amount of electromagnetic radiation that is transmitted through or reflected from each pixel may be controlled by how long the pixel is in the on or off position while the electromagnetic radiation source is activated. The resolution of the treatment beam and intensity at each point is controlled within the resolution of the beam shaping mechanism 318, although not limited to this embodiment.

Referring now to FIG. 8B, shown is another schematic block diagram of an embodiment of the main control system. Once the operator decides that the treatment beam parameters and shape are correct, the operator may initiate the treatment beam via the user interface 314. The treatment source 322, which can be a laser, LED, flash lamp, or other device, is activated. The electromagnetic radiation 326 from the treatment source 322 may pass through collimating optics 324 and the electromagnetic radiation 327 from the collimating optics 324 may pass into the beam shaping mechanism 318. The pixels that are turned on reflect or transmit electromagnetic radiation 330 through the projection optics 328. The projection optics 328 are designed to transfer the image of the beam shaping optics 318 to the proximal end of the electromagnetic radiation delivery device 304.

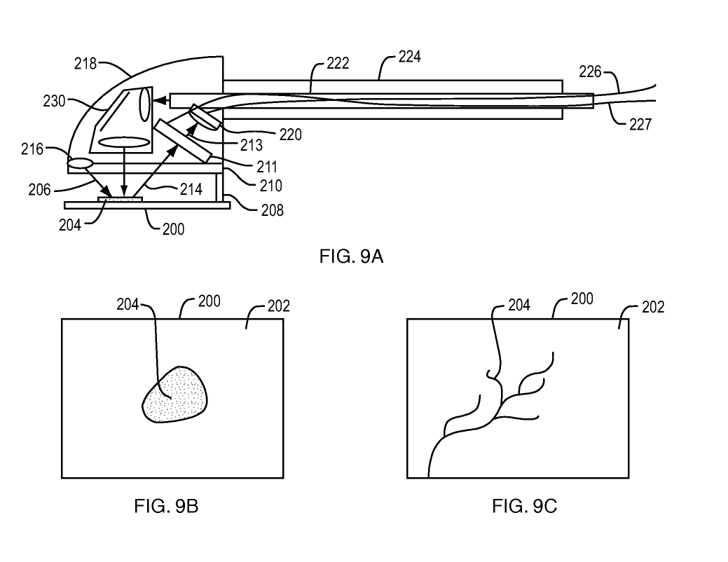

Referring now to FIG. 9A, shown is a schematic illustration of another embodiment of the handpiece. FIGS. 9B and 9C are schematic illustrations of skin abnormalities. The treatment electromagnetic radiation travels through the electromagnetic radiation guide 222 and enters the beam steering optics 230 in the treatment head 218. The electromagnetic radiation 206 exiting the treatment head 218 passes through the window 210 and onto the treatment area 200. The control electronics 308 (shown in FIG. 8B) allow the system to produce a treatment beam that has a predetermined border around the lesion. For photodynamic therapy treatments, or treatments in which the treatment electromagnetic radiation is applied for multiple seconds (or longer), the system will continually monitor the tissue and adjust the treatment beam characteristics accordingly.

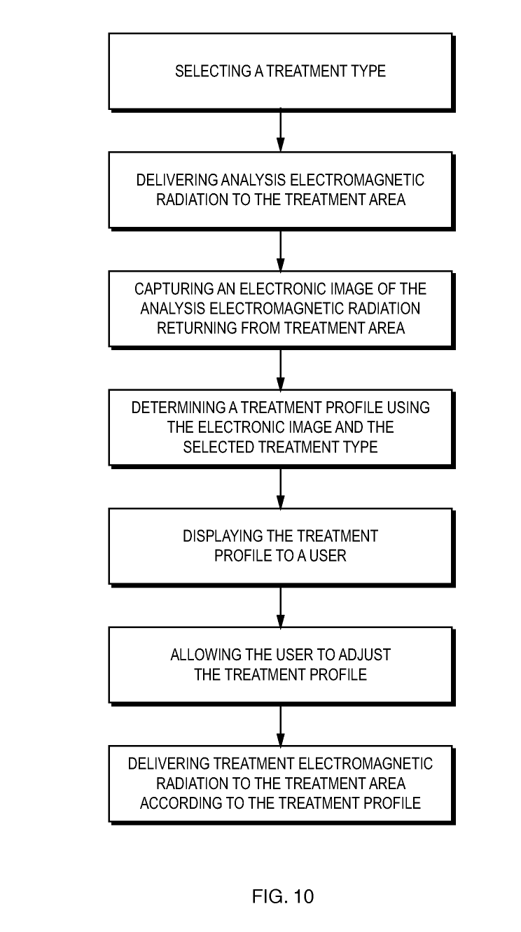

Referring now to FIG. 10, shown is a flowchart depicting one embodiment of the method of dermatological treatment. The following steps may be performed, although not limited thereto: (a) selecting a treatment type; (b) delivering analysis electromagnetic radiation to the treatment area; (c) capturing an electronic image of the analysis electromagnetic radiation returning from treatment area; (d) determining a treatment profile using the electronic image and the selected treatment type; (e) displaying the treatment profile to a user; (f) allowing the user to adjust the treatment profile; and (g) delivering treatment electromagnetic radiation to the treatment area according to the treatment profile.

In one embodiment, the method for dermatological treatment may have analysis electromagnetic radiation in more than one wavelength or waveband.

In one embodiment, the method for dermatological treatment may further comprise a handpiece that delivers the treatment electromagnetic radiation. The handpiece may also capture the electronic image, although not limited thereto.

In one embodiment, the method for dermatological treatment may repeat steps (b)-(d) to update the treatment profile in substantially real-time.

In one embodiment, the method for dermatological treatment may have a pixilated treatment profile with threshold values for each pixel

In one embodiment, the method for dermatological treatment may have pixels that are within threshold values turn to a specific color as the user adjusts the treatment profile to match the treatment area.

In some embodiments, the method for dermatological treatment may perform, although not limited thereto, tattoo removal, vascular lesion removal, skin lesion repair, acne removal, and hair removal.

In one embodiment, although not limited thereto, the system may have user controls so that a user may first select the type of treatment he or she desires to deliver to the patient before initializing treatment. Such a setting may help to determine treatment thresholds, discussed further below. User controls may be located on a control panel on the main control system or may be integrated into a touch screen display, although not limited thereto. After selecting the type of treatment, the user may place the handpiece over the treatment area and check the target (e.g., abnormality, etc.) orientation on the display to ensure it is properly centered under the treatment head.

The system may then deliver electromagnetic radiation to analyze the treatment area and development of a treatment profile. Electromagnetic radiation in one or more wavebands (e.g., white light source, LEDs, infrared, etc.) may be used. In one embodiment, an agent may first be added to the treatment area which absorbs electromagnetic radiation at certain frequencies and then produces a fluorescence signal.

An electronic image showing the effects of the electromagnetic radiation on the treatment area (e.g., reflection, absorption, fluorescence, etc.) may then be acquired by an image acquisition system. A sensor array such as one found in a charge-coupled device (CCD) camera may create an image of the treatment area. The image may first be filtered using a polarizer, band pass filter, or long pass filter, although not limited thereto. Filters may be employed to narrow the band of the reflected electromagnetic radiation received by the system. For example, although not limited thereto, long pass filters may be used if measuring fluorescent light.

The acquired image may have information about the treatment area at several wavelengths (e.g., 460 nm, 525 nm, 630 nm and 800 nm, etc.) which the system may use to determine tissue color and/or other tissue properties. Tissue properties include, but are not limited to, hemoglobin content, collagen, lipid content, or temperature. The image of the treatment area, which may be pixilated, although not limited thereto, may be translated into intensity values for each pixel. In one embodiment, the output from a CCD camera (e.g., sensor array, etc.) may be in the form of data numbers (DN) where the maximum value depends upon the camera bit depth. For example, although not limited thereto, a 12-bit CCD camera may yield 4096 levels of resolution.

The system may then automatically set treatment thresholds for pixels above certain intensity values at each of the various wavelengths or wavebands. Based on factors including the treatment type, wavelength or waveband, intensity level of reflected electromagnetic radiation, and skin type, initial thresholds can be set by a percentage of the maximum value, although not limited thereto. The system may use a histogram of intensities values of the analysis electromagnetic radiation to determine which pixel values are near the baseline, or normal skin tone values. The baseline of normal skin color or tone will be determined using the histogram. Using histogram data, threshold values may be set at each pixel to create a treatment profile. The threshold values may be above specific value(s) in the various wavebands, below specific values within the various wavebands, or could be a range of values within the various wavebands.

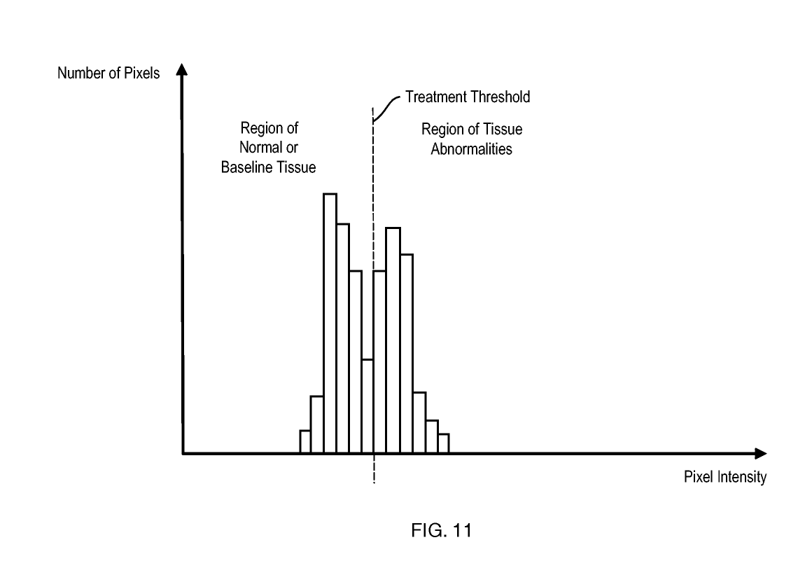

Referring now to FIG. 11, shown is a graphical representation of one embodiment of a histogram.

A threshold is determined such that any pixel with an intensity level lower (or the skin is darker) than the threshold gets treated, although not limited thereto. Thresholds can be set for color, which can be a determinant for whether or not a part of an abnormality has been treated. For example, although not limited thereto, a particular wavelength of electromagnetic radiation may also be used to measure relative temperature, where an increase in temperature indicates treatment has occurred and the relative amount of electromagnetic radiation that was absorbed by the tissue. In fact, the system may image the treatment area using any number of wavelengths of electromagnetic radiation in order to obtain any number of particular attributes about the treatment area and it is not limited to color.

Treatment thresholds may be set by the intensity of the RGB (Red-Green-Blue) components, although not limited thereto. An RGB CCD camera by itself can provide the RGB values at each pixel in the image of the treatment area, or a filter wheel can be used to filter for specific wavebands or wavelengths. Since there is so much variation in the population's skin color, common thresholds for various types of skin may be stored in a histogram. The following values provide a starting treatment threshold, although not limited thereto: dark skin (R-G-B, 94-28-13), light skin (R-G-B, 241-149-108). The operator can then adjust the RGB values up and down for the specific patient.

Pixilation allows the customization of a treatment profile since a non-uniform lesion (see, for example, FIGS. 3A and 3B) may have varying color, or other measurable tissue properties. A non-uniform pigmented lesion may vary in color and darker portions may require different treatment than lighter portions. As an example, although not limited thereto, the maximum intensity (e.g., brightness) of a pixel may be X and the threshold may be set to Y. Any part of the treatment area that has an intensity less than or equal to Y will be treated. The treatment profile may also provide lighter areas with more treatment light since darker areas absorb more light and lighter areas are less sensitive.

Different thresholds can be set for different ranges of pixel intensity, or the treatment intensity can vary continuously throughout the lesion. For instance, if the analysis electromagnetic radiation of the lesion has a continuum of values, the corresponding pixel values may also be a continuum to optimize the treatment of the lesion.

The system may then display the treatment profile (for example, comprising threshold values for each pixel, although not limited thereto) to the user on a display. The treatment profile may overlay the image of the displayed treatment area on the display and the user may operate controls to adjust the thresholds in relation to the treatment area, although not limited thereto. If the user does not choose to override the automatically calculated treatment profile, then the user may initiate treatment. Otherwise, the user may utilize user controls (e.g., control panel buttons, knobs, sliders, touch screen, etc.) to adjust the threshold values. For example, pixels that are within a given threshold may be set to a specific color such as blue while all other pixels may be given their actual color, although not limited thereto. All pixels within the threshold may then turn blue as the threshold is adjusted to match the treatment profile to the treatment area. This allows the user to ensure that the treatment beam profile automatically created by the system is appropriate, and allows the user to adjust the treatment profile as necessary. Once the blue colored pixels span the entire abnormality, for example, the treatment profile may treat only the abnormality. Providing the operator a view of the treatment area permits the safety of manual control over the treatment settings.

The use of thresholds allow for an additional safety mechanism. While not required, many dermatologists are cautious and may desire this functionality. The use of thresholds also provides user flexibility if the user wants to treat beyond the surface borders of an abnormality.

Once the treatment beam has been determined, the treatment may be delivered to the treatment area. Some common ranges of wavelengths and intensities for some treatment profiles include, although not limited thereto: laser wavelengths of 532 nm, 585 nm, 755 nm, 1064 nm, 2940 nm, 10.6 nm; intense pulsed light waveband of 515-1200 nm; energy ranges of 1 J-1000 J; and pulse lengths of 0.1-1000 ms.

Pixilation of the treatment beam may be accomplished by using a beam shaping mechanism, although not limited thereto. A beam shaping mechanism may be a digital micromirror device (DMD), although not limited thereto. The image may initially come from the image acquisition system, but then may go through the threshold algorithm for adjustment and back to the DMD for delivery of the treatment electromagnetic radiation. The treatment may hit an array of pixels that are essentially turned on and off according to the intensity level needed. For instance, if the intensity required for one particular pixel is ½ of maximum, then that pixel may be turned on for ½ of the time. In one embodiment of a DMD device, although not limited thereto, the pixels are mirrors that are either turned 12 degrees to send the light through, or rotated −12 degrees to send the light away from the treatment area and into an absorber. The pixels may be approximately 13 microns in diameter, although not limited thereto. The treatment electromagnetic radiation may also be adjusted by changing the power of the lenses in the handpiece.

Most aesthetic dermatological treatments may be delivered in less than 1 second, although not limited thereto. The system may provide an audible alarm or other notification indicating that treatment is complete. The system may also check for post treatment indications and the user can leave the handpiece over the treatment area so that the system analyzes the treatment area again to see if full treatment was successfully delivered. If an area was not efficiently treated, it may be re-treated. In one embodiment, post treatment thresholds may be set automatically after treatment. The post treatment thresholds may look for post treatment indications such as relative tissue temperature or an increase in tissue blood content, and analysis may continue in real-time to read the tissue information. If the user is satisfied with the treatment at a first location, the user may move the handpiece to a new location and begin treatment again. The user may also change the treatment type if necessary.

While the present teachings have been described above in terms of specific embodiments, it is to be understood that they are not limited to these disclosed embodiments. Many modifications and other embodiments will come to mind to those skilled in the art to which this pertains, and which are intended to be and are covered by both this disclosure and the appended claims. It is intended that the scope of the present teachings should be determined by proper interpretation and construction of the appended claims and their legal equivalents, as understood by those of skill in the art relying upon the disclosure in this specification and the attached drawings.

I/we claim:

1. A system for dermatological treatment of a treatment area, comprising:
an analysis electromagnetic radiation system that delivers analysis electromagnetic radiation to the treatment area;
an image acquisition system that acquires an electronic image of the analysis electromagnetic radiation delivered to the treatment area;
computer readable media having threshold treatment values;
control electronics that creates a treatment profile by selecting threshold treatment values from the computer readable media using the electronic image;
a display that displays the treatment profile; and
a treatment electromagnetic radiation system that delivers treatment electromagnetic radiation to the treatment area according to the treatment profile.

2. The system of claim 1 wherein the analysis electromagnetic radiation is in more than one wavelength.

3. The system of claim 1 further comprising a filter adjacent to the image acquisition system.

4. The system of claim 1 further comprising a handpiece that delivers the treatment electromagnetic radiation.

5. The system of claim 4 wherein the handpiece also acquires the electronic image.

6. The system of claim 1 further comprising user controls for selecting treatment type.

7. The system of claim 1 further comprising user controls for adjusting the treatment profile and wherein the display also displays the treatment area.

8. The system of claim 1 further comprising a beam shaping mechanism that shapes the treatment electromagnetic radiation.

9. The system of claim 1 wherein the treatment profile is determined using the wavelength intensity of the electronic image.

10. The system of claim 9 wherein the treatment profile is pixilated and comprises threshold values for each pixel.

11. The system of claim 1 wherein the image acquisition system comprises a sensor array.

12. The system of claim 1 wherein the threshold values are stored in histogram.

13. The system of claim 1 further comprising computer readable media for storing the electronic image.

14. The system of claim 1 wherein the image acquisition system acquires an electronic image after treatment is initiated and the control electronics updates the treatment profile substantially in real-time.

15. A method of dermatological treatment of a treatment area, comprising the steps of:
(a) selecting a treatment type;
(b) delivering analysis electromagnetic radiation to the treatment area;
(c) capturing an electronic image of the analysis electromagnetic radiation returning from treatment area;
(d) determining a treatment profile using the electronic image and the selected treatment type;
(e) displaying the treatment profile to a user;
(f) allowing the user to adjust the treatment profile; and
(g) delivering treatment electromagnetic radiation to the treatment area according to the treatment profile.

16. The method of claim 15 wherein the analysis electromagnetic radiation is in more than one wavelength.

17. The method of claim 15 further comprising a handpiece that delivers the treatment electromagnetic radiation

18. The method of claim 17 wherein the handpiece also captures the electronic image.

19. The method of claim 15 wherein steps (b)-(d) are repeated to update the treatment profile in substantially real-time.

20. The method of claim 15 wherein the treatment profile is pixilated and comprises threshold values for each pixel.

21. The method of claim 20 wherein pixels within threshold values turn to a specific color as the user adjusts the treatment profile to match the treatment area.

22. The method of claim 15 wherein the dermatological treatment comprises tattoo removal.

23. The method of claim 15 wherein the dermatological treatment comprises vascular lesion removal.

24. The method of claim 15 wherein the dermatological treatment comprises skin lesion repair.

25. The method of claim 15 wherein the dermatological treatment comprises acne removal.

26. The method of claim 15 wherein the dermatological treatment comprises hair removal.
